(19) United States
(12) Patent Application Publication
(10) Pub. No.: US 2024/0269233 A1
(43) Pub. Date: Aug. 15, 2024

(54) METHODS FOR TREATMENT OF OSTEOARTHRITIS

(71) Applicant: Novartis AG, Basel (CH)

(72) Inventor: Celeste SCOTTI, Basel (CH)

(21) Appl. No.: 18/562,075

(22) PCT Filed: May 23, 2022
(86) PCT No.: PCT/IB2022/054816

§ 371 Date: Nov. 17, 2023

Related U.S. Application Data
(60) Provisional application No. 63/192,348, filed on May 24, 2021.

(51) Int. Cl. A61K 38/18; A61K 9/00; A61P 19/02; C07K 14/515

(52) U.S. Cl. CPC A61K 38/1891; A61K 9/0019; A61P 19/02; C07K 14/515

(57) ABSTRACT

Provided herein are methods and dosage regimens for the treatment of osteoarthritis (e.g., knee osteoarthritis). These methods and dosage regimens include intra-articular injections of Compound 1.

### SEQUENCE LISTING

The instant application contains a Sequence Listing which has been submitted electronically in ASCII format and is hereby incorporated by reference in its entirety. Said ASCII copy, created on May 18, 2022, is named PAT059120-WO-PCT_SL.txt and is 2,266 bytes in size.

### TECHNICAL FIELD

The disclosure relates to methods, treatment regimens, uses, kits and therapies for treating osteoarthritis by administering a therapeutic polypeptide according to the dosing regimens disclosed herein.

### BACKGROUND OF THE INVENTION

Osteoarthritis (OA), a slowly progressive disease with a multifactorial pathophysiology, is one of the most common chronic health conditions in adults, and a leading cause of pain and disability (OARSI 2016, submitted to the U.S. Food and Drug Administration, viewed 16 Dec. 2019, p. 1-103). Due to the demographic changes in an ageing society and an increase in the incidence of obesity, the prevalence of OA will steadily increase, posing a significant burden on global health-care systems. (OARSI 2016; Fu & Griffin 2014, Biomaterials, vol 16. Springer, Ch). On the individual level OA leads to suffering with OA symptoms being responsible for 6.8% of overall DALYs (disability adjusted life years). (Cross et al 2014, Ann. Rheum. Dis. 73, 1323-1330; Kassebaum et al 2016, Lancet 388, 1603-1658). An excess mortality rate from cardiovascular events also has been observed in association with OA. (Kloppenburg & Berenbaum 2019, Osteoarthr. Cartil. 28, 242-248). Finally, OA is a relevant financial risk factor with loss of work days and out of pocket expenditure. (Puig-Junoy & Zamora 2015, Semin. Arthritis Rheum. 44, 531-541; Sharif et al 2015, Osteoarthr. Cartil. October 23 (10):1654-63).

The knee joint is the most common weight bearing joint affected by OA. Current medical treatments of OA focus on addressing pain, but there are no disease-modifying OA drugs (DMOADs), e.g., chondro-anabolic treatments, available to induce cartilage regeneration. (Lohmander and Roos 2019, Nat Rev Rheumatol p. 133-135). Eventual joint failure requiring surgical joint replacement is common, with over a million such operations annually in the United States (Williams et al 2015, NCHS Data Brief p. 1-8; Wolford et al 2015, NCHS Data Brief p. 1-8). However, not all patients are satisfied with the result, or benefit from joint replacement surgery. In a long-term outcome study in an OA cohort with a high prevalence of multiple affected joints and comorbidities, only half of those who received joint replacement achieved a good surgical outcome, defined as improved pain and reduced disability. (Hawker et al 2013, Arthritis Rheum. 65(5):1243-52).

### SUMMARY OF THE INVENTION

The present invention provides methods for treating osteoarthritis (e.g., knee osteoarthritis) by administering therapeutically effective amounts of a modified human ANGPTL3 polypeptide, i.e., Compound 1, to the joint (e.g., knee) of a human subject according to the dosing regimens disclosed herein. The inventors have surprisingly discovered that administering Compound 1 according to the presently described dosing regimens results in a therapeutically beneficial effect for a human subject despite administering fewer injections on a less frequent basis than what has been previously described.

In some embodiments, the present invention provides for a method of treating osteoarthritis comprising administering to a human subject in need thereof one or more doses of a therapeutically effective amount of Compound 1 by intra-articular injection to a joint of the subject according to a dosing regimen comprising one or more dosing cycles. In certain embodiments, the osteoarthritis is knee osteoarthritis and the joint is the knee joint. In some embodiments, the dosing cycle is a six-month dosing cycle comprising one intra-articular injection. In other embodiments, the dosing cycle is a six-month dosing cycle comprising three intra-articular injections per dosing cycle with one injection administered once a month for three consecutive months. In some embodiments, the dosing regimen comprises two six-month dosing cycles, four six-month dosing cycles, or eight six-month dosing cycles, wherein each dosing cycle may comprise one intra-articular injection per dosing cycle or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In some embodiments, the present invention provides for a method of treating osteoarthritis comprising administering to a human subject in need thereof one or more doses of a therapeutically effective amount of Compound 1 by intra-articular injection to a joint of the subject according to a dosing regimen comprising one or more twelve-month dosing cycles. In certain embodiments, the osteoarthritis is knee osteoarthritis and the joint is the knee joint. In some embodiments, the twelve-month dosing cycle comprises one intra-articular injection. In other embodiments, the twelve-month dosing cycle comprises three intra-articular injections per dosing cycle with one injection administered once a month for three consecutive months. In some embodiments, the dosing regimen comprises two twelve-month dosing cycles, or four twelve-month dosing cycles, wherein each dosing cycle may comprise one intra-articular injection or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In some embodiments, treatment according to one of the described dosing regimens results in an increase in cartilage thickness compared to placebo as determined by quantitative magnetic resonance imaging. In other embodiments, the increase in cartilage thickness is in the targeted knee joint. In some embodiments, treatment according to one of the described dosing regimens results in a decrease in osteoarthritis pain in the target knee joint compared to placebo as determined by WOMAC pain and WOMAC pain walking on flat surface item. In other embodiments, treatment according to one of the described dosing regimens results in an increase in physical function in the target knee joint compared to placebo as determined by WOMAC function. In other embodiments, treatment according to one of the described dosing regimens results in an increase in performance-based physical function in the target knee joint compared to placebo as determined by a 40-meter (4×10 m) fast-paced walk test, 30-second chair stand test, and 6-minute walking test. In other embodiments, treatment according to one of the described dosing regimens results in an increase in structural progression of the target knee joint compared to placebo as determined by qMRI and x-ray.

In other embodiments, the present invention provides Compound 1 for use in the treatment of osteoarthritis via administration to a human subject in need thereof one or more doses of a therapeutically effective amount of Compound 1 by intra-articular injection to a joint of the subject according to a dosing regimen comprising one or more dosing cycles. In certain embodiments, the osteoarthritis is knee osteoarthritis and the joint is the knee joint. In some embodiments, the dosing cycle is a six-month dosing cycle comprising one intra-articular injection. In other embodiments, the dosing cycle is a six-month dosing cycle comprising three intra-articular injections per dosing cycle with one injection administered once a month for three consecutive months. In some embodiments, the dosing regimen comprises two six-month dosing cycles, four six-month dosing cycles, or eight six-month dosing cycles, wherein each dosing cycle may comprise one intra-articular injection or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In other embodiments, the present invention provides Compound 1 for use in the treatment of osteoarthritis via administration to a human subject in need thereof one or more doses of a therapeutically effective amount of Compound 1 by intra-articular injection to a joint of the subject according to a dosing regimen comprising one or more twelve-month dosing cycles. In certain embodiments, the osteoarthritis is knee osteoarthritis and the joint is the knee joint. In some embodiments, the twelve-month dosing cycle comprises one intra-articular injection. In other embodiments, the twelve-month dosing cycle comprises three intra-articular injections per dosing cycle with one injection administered once a month for three consecutive months. In some embodiments, the dosing regimen comprises two twelve-month dosing cycles, or four twelve-month dosing cycles, wherein each dosing cycle may comprise one intra-articular injection or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In some embodiments, Compound 1 for use according to one of the described dosing regimens results in an increase in cartilage thickness compared to placebo as determined by quantitative magnetic resonance imaging. In other embodiments, the increase in cartilage thickness is in the targeted knee joint. In some embodiments, Compound 1 for use according to one of the described dosing regimens results in a decrease in osteoarthritis pain in the target knee joint compared to placebo as determined by WOMAC pain and WOMAC pain walking on flat surface item. In other embodiments, Compound 1 for use according to one of the described dosing regimens results in an increase in physical function in the target knee joint compared to placebo as determined by WOMAC function. In other embodiments, Compound 1 for use according to one of the described dosing regimens results in an increase in performance-based physical function in the target knee joint compared to placebo as determined by a 40-meter (4×10 m) fast-paced walk test, 30-second chair stand test, and 6-minute walking test. In other embodiments, Compound 1 for use according to one of the described dosing regimens results in an increase in structural progression of the target knee joint compared to placebo as determined by qMRI and x-ray.

In other embodiments, the present invention provides Compound 1 for use in the manufacture of a medicament for the treatment of osteoarthritis via administration to a human subject in need thereof one or more doses of a therapeutically effective amount of Compound 1 by intra-articular injection to a joint of the subject according to a dosing regimen comprising one or more dosing cycles. In certain embodiments, the osteoarthritis is knee osteoarthritis and the joint is the knee joint. In some embodiments, the dosing cycle is a six-month dosing cycle comprising one intra-articular injection. In other embodiments, the dosing cycle is a six-month dosing cycle comprising three intra-articular injections per dosing cycle with one injection administered once a month for three consecutive months. In some embodiments, the dosing regimen comprises two six-month dosing cycles, four six-month dosing cycles, or eight six-month dosing cycles, wherein each dosing cycle may comprise one intra-articular injection or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In other embodiments, the present invention provides Compound 1 for use in the manufacture of a medicament for the treatment of osteoarthritis via administration to a human subject in need thereof one or more doses of a therapeutically effective amount of Compound 1 by intra-articular injection to a joint of the subject according to a dosing regimen comprising one or more twelve-month dosing cycles. In certain embodiments, the osteoarthritis is knee osteoarthritis and the joint is the knee joint. In some embodiments, the twelve-month comprises one intra-articular injection. In other embodiments, the twelve-month dosing cycle comprises three intra-articular injections per dosing cycle with one injection administered once a month for three consecutive months. In some embodiments, the dosing regimen comprises two twelve-month dosing cycles, or four twelve-month dosing cycles, wherein each dosing cycle may comprise one intra-articular injection or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In some embodiments, Compound 1 for use in the manufacture of a medicament for the treatment of osteoarthritis when administered to a human subject in need thereof according to one of the described dosing regimens results in an increase in cartilage thickness compared to placebo as determined by quantitative magnetic resonance imaging. In other embodiments, the increase in cartilage thickness is in the targeted knee joint. In some embodiments, Compound 1 for use according to one of the described dosing regimens results in a decrease in osteoarthritis pain in the target knee joint compared to placebo as determined by WOMAC pain and WOMAC pain walking on flat surface item. In other embodiments, Compound 1 for use according to one of the described dosing regimens results in an increase in physical function in the target knee joint compared to placebo as determined by WOMAC function. In other embodiments, Compound 1 for use according to one of the described dosing regimens results in an increase in performance-based physical function in the target knee joint compared to placebo as determined by a 40-meter (4×10 m) fast-paced walk test, 30-second chair stand test, and 6-minute walking test. In other embodiments, Compound 1 for use according to one of the described dosing regimens results in an increase in structural progression of the target knee joint compared to placebo as determined by qMRI and x-ray.

In certain embodiments, the present invention provides a pharmaceutical composition comprising Compound 1 for use in the treatment of osteoarthritis via administration to a human subject in need thereof one or more doses of a therapeutically effective amount of Compound 1 by intra-articular injection to a joint of the subject according to a dosing regimen comprising one or more dosing cycles. In certain embodiments, the osteoarthritis is knee osteoarthritis and the joint is the knee joint. In some embodiments, the dosing cycle is a six-month dosing cycle comprising one intra-articular injection. In other embodiments, the dosing cycle is a six-month dosing cycle comprising three intra-articular injections per dosing cycle with one injection administered once a month for three consecutive months. In some embodiments, the dosing regimen comprises two six-month dosing cycles, four six-month dosing cycles, or eight six-month dosing cycles, wherein each dosing cycle may comprise one intra-articular injection or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In other embodiments, the present invention provides a pharmaceutical composition comprising Compound 1 for use in the treatment of osteoarthritis via administration to a human subject in need thereof one or more doses of a therapeutically effective amount of Compound 1 by intra-articular injection to a joint of the subject according to a dosing regimen comprising one or more twelve-month dosing cycles. In certain embodiments, the osteoarthritis is knee osteoarthritis and the joint is the knee joint. In some embodiments, the twelve-month dosing cycle comprises one intra-articular injection. In other embodiments, the twelve-month dosing cycle comprises three intra-articular injections per dosing cycle with one injection administered once a month for three consecutive months. In some embodiments, the dosing regimen comprises two twelve-month dosing cycles, or four twelve-month dosing cycles, wherein each dosing cycle may comprise one intra-articular injection or three intra-articular injections per dosing cycle with one injection administered once a month for three consecutive months. In certain embodiments, the therapeutically effective amount of Compound 1 administered per injection is 20 mg. In other embodiments, the therapeutically effective amount of Compound 1 administered per injection is 40 mg.

In some embodiments, a pharmaceutical composition comprising Compound 1 for use in the treatment of osteoarthritis when administered to a human subject in need thereof according to one of the described dosing regimens results in an increase in cartilage thickness compared to placebo as determined by quantitative magnetic resonance imaging. In other embodiments, the increase in cartilage thickness is in the targeted knee joint. In some embodiments, Compound 1 for use according to one of the described dosing regimens results in a decrease in osteoarthritis pain in the target knee joint compared to placebo as determined by WOMAC pain and WOMAC pain walking on flat surface item. In other embodiments, Compound 1 for use according to one of the described dosing regimens results in an increase in physical function in the target knee joint compared to placebo as determined by WOMAC function. In other embodiments, Compound 1 for use according to one of the described dosing regimens results in an increase in performance-based physical function in the target knee joint compared to placebo as determined by a 40-meter (4×10 m) fast-paced walk test, 30-second chair stand test, and 6-minute walking test. In other embodiments, Compound 1 for use according to one of the described dosing regimens results in an increase in structural progression of the target knee joint compared to placebo as determined by qMRI and x-ray.

Various aspects of the disclosure are described herein and in the claims.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise. Although methods and materials similar or equivalent to those described herein can be used in the practice or testing of the disclosure, suitable methods and materials are described below. All publications, patent applications, patents, and other references mentioned herein are incorporated by reference in their entireties for all purposes. The references cited herein are not admitted to be prior art to the claimed disclosure. In the case of conflict, the present specification, including definitions, will control. In addition, the materials, methods, and examples are illustrative only and are not intended to be limiting.

Other features and advantages of compounds, compositions, and methods disclosed herein will be apparent from the following detailed description and claims

### DESCRIPTION OF THE DRAWINGS

FIG. 1 is a schematic representation of exemplary dosing regimens. CMPD1=Compound 1.

FIG. 2 depicts immunohistochemical staining of osteochondral knee tissue obtained during a total knee replacement demonstrating that Compound 1 has a more pronounced penetration in (A) damaged cartilage tissue versus (B) undamaged cartilage tissue.

FIG. 3 depicts RNA-Seq analysis of cartilage biopsies harvested from worn and unworn areas of the knee demonstrates that following intra-articular injection of Compound 1: A) 151 genes were significantly up- or down-regulated in response to Compound 1 as compared to placebo, and B) several genes involved in cartilage homeostasis and repair are modulated by Compound 1 up to 21 days post-injection. CMPD1=Compound 1.

FIG. 4A) a cartilage 3D MRI rendering showing administration of a single intra-articular injection of Compound 1 to an exemplary patient undergoing autologous chondrocyte implantation resulted in an increased filling of a surgically created lesion at the donor site as compared to placebo. B) is a graphical representation demonstrating that percent refilling of the donor site is increased over time in an exemplary patient administered a single intra-articular injection of Compound 1 as compared to placebo. CMPD1=Compound 1.

FIG. 5A) Sodium-MRI images confirming the hyaline-like cartilage nature of regenerated tissue at the donor site of an exemplary patient undergoing autologous chondrocyte implantation that was administered a single intra-articular injection of Compound 1 at 2 time points as compared to placebo. B) demonstrates that the treatment response calculated as percent change from baseline in sodium signal intensity in the donor site corrected by sodium signal intensity in the reference region of an exemplary patient administered Compound 1 increases over time as compared to placebo. CMPD1=Compound 1.

FIG. 6 depicts 3D MRI images from an exemplary patient treated with four weekly intra-articular injections of Compound 1 demonstrating successful articular cartilage lesion filling. CMPD1=Compound 1.

FIG. 7 depicts the model estimated percentage difference of Compound 1 to placebo in cartilage defect volume over time (PD analysis set) in patients with partial thickness lesions demonstrating that Compound 1 decreases the cartilage defect volume. A) overall and B) in femur. MMRM=mixed model repeated measures; CMPD1=Compound 1.

### DETAILED DESCRIPTION OF THE INVENTION

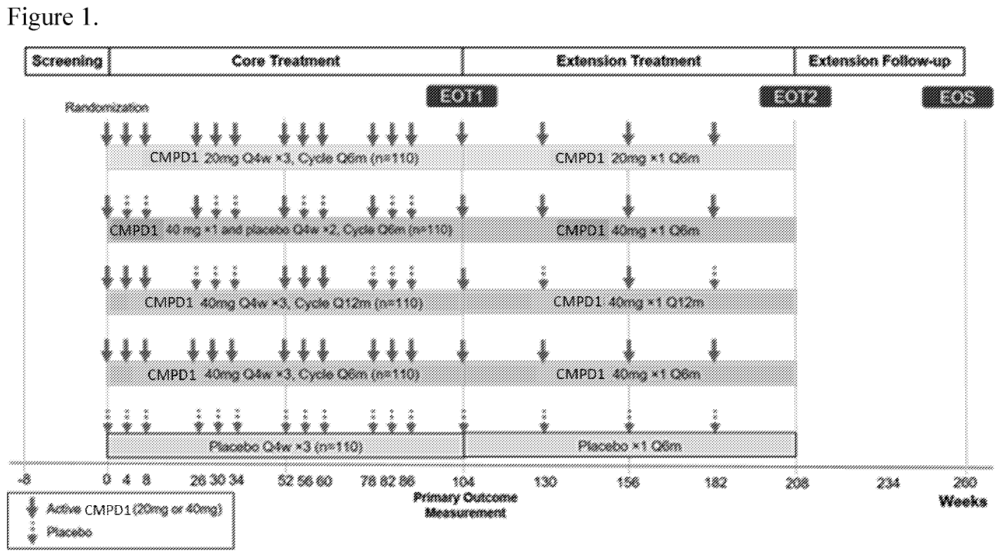

Modified human ANGPTL3 polypeptides have been shown to demonstrate chondrogenic and chondroprotective effects. Examples of such polypeptides have been previously described in WO2014/138687, the contents of which are fully incorporated by reference. Methods of administering such polypeptides for the purpose of treating cartilage damage and/or arthritis have been previously described in WO2018/087727. Prior art methods of administering Compound 1, e.g., those described in WO2018/087727, contemplate frequent intra-articular injections (e.g., weekly) and do not describe defined dosing and resting periods. Instead, the prior art methods contemplate continuing the administration of Compound 1 until such time as the condition, e.g., cartilage damage or arthritis, is treated. Although such methods may be effective, there remains a need to establish dosing regimens that convey a therapeutic benefit while minimizing the number of intra-articular injections. The present inventors have now met that need with the surprising discovery that Compound 1 may be administered on a less frequent basis than was previously thought while still conveying a therapeutically effective benefit. (FIG. 1). Administration according to the dosing regimens described herein also results in fewer total injections of Compound 1. Due to the lower number and reduced frequency of intra-articular injections, the inventive dosing regimens also reduce patient discomfort, minimize the potential for inadvertent structural damage to the joint during administration, lower the risk for adverse events associated with intra-articular injection, e.g., injection site infection, and increase patient compliance.

### Definitions

The term “subject” refers to an animal, human or non-human, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs.

The term “treatment”, “treating,” or “treat” is herein defined as both therapeutic treatment and prophylactic or preventive measures, wherein the object is to prevent or slow down an undesired physiological change or disorder. For purpose of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and reversal (whether partial or total), whether detectable or undetectable.

A subject is “in need of” a treatment if such subject would benefit biologically, medically or in quality of life from such treatment.

The term “pharmaceutically acceptable” as used herein refers to those compounds, materials, compositions and/or dosage forms, which are, within the scope of sound medical judgment, suitable for contact with the tissues of a subject, e.g., a mammal or human, without excessive toxicity, irritation, allergic response and other problems or complications commensurate with a reasonable benefit/risk ratio.

The term “administration” or “administering” of the subject compound means providing a drug, a modified derivative of a drug, or a prodrug to a subject in need of treatment.

The term “dosing regimen,” as used herein, refers to the treatment plan specifically indicating the administration pattern of a drug over a period of time. The dosing regimen defines the amount of a drug and the number and frequency of its administrations that is employed in the treatment of a disease. The dosing regimens of the present invention may comprise one or more dosing cycles.

The term “dosing cycle,” as used herein, means administering a drug for a period of time (i.e., the dosing period) followed by a resting period before administration of the drug is resumed. A dosing cycle begins with the first administration of the drug in that cycle. The term “resting period,” as used herein, refers to a period of time during which the subject is not given a drug (i.e., a period of time wherein the treatment with a drug is withheld). For example, if a drug is given on a daily, weekly, or monthly basis, there would be rest period if the administration is discontinued for some time, e.g., for some number of days, weeks, or months. The dosing period and/or the resting period of the dosing cycle can be the same or different between cycles. For example, if the dosing period is once weekly the resting period may be one week or more than one week. It is contemplated that the dose of the drug administered can be the same or different between cycles.

The term “dose” refers to a specified amount of a drug administered at one time. As used herein, the dose is the amount of the drug that elicits a therapeutic effect.

The term “a therapeutically effective amount” of a drug refers to an amount of a drug that will elicit the desired biological or medical response in a subject, for example, at least partially ameliorate symptoms, alleviate conditions, slow or delay progression, or reverse a disorder or disease.

As used herein, the terms “a” and “an” and “the” and similar references in the context of describing the invention are to be construed to cover both the singular and the plural, unless otherwise indicated herein or clearly contradicted by context. Where the plural form is used for compounds, salts, and the like, this is taken to mean also a single compound, salt, or the like.

The term “or” is used herein to mean, and is used interchangeably with, the term “and/or”, unless context clearly indicates otherwise.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values. When describing a dosage herein as “about” a specified amount, the actual dosage can vary by up to 10% from the stated amount: this usage of “about” recognizes that the precise amount in a given dosage form may differ slightly from an intended amount for various reasons without materially affecting the in vivo effect of the administered compound. When describing a dosage herein as a specified amount, i.e. without the term “about”, the actual dosage can vary by up to 10% (preferably by up to 5%) from the stated amount: this usage recognizes that the precise amount in a given dosage form may differ slightly from an intended amount for various reasons without materially affecting the in vivo effect of the administered compound.

The terms “comprising” and “including” are used herein in their open-ended and non-limiting sense unless otherwise noted.

By “a combination” or “in combination with” it is not intended to imply that the therapy or the therapeutic agents must be physically mixed or administered at the same time and/or formulated for delivery together, although these methods of delivery are within the scope described herein. A therapeutic agent in these combinations can be administered concurrently with, prior to, or subsequent to, one or more other additional therapies or therapeutic agents. The therapeutic agents can be administered in any order. In general, each agent will be administered at a dose and/or on a time schedule determined for that agent. It will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized as single-agent therapeutics.

The combinations of the invention have therapeutic or protective functions or both. For example, these molecules may be administered to a human subject, to treat and/or prevent a variety of disorders, such as OA as described herein.

The term “combination” as used herein refers to either a fixed combination in one dosage unit form, or non-fixed combination, or a kit of parts for the combined administration where two or more therapeutic agents may be administered together, independently at the same time or separately within time intervals, especially where these time intervals allow that the combination partners show a cooperative, e.g., synergistic, effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., different i.a. formulations, or formulations for different routes of administration) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

By simultaneous therapeutic use, within the meaning of the present invention is meant an administration of at least two active ingredients by the same route and at the same time or at substantially the same time.

By separate use, within the meaning of the present invention is meant in particular an administration of at least two active ingredients at the same time or at substantially the same time by different routes.

By sequential therapeutic use is meant administration of at least two active ingredients at different times, the administration route being identical or different. More particularly by an administration method is meant according to which the whole administration of one of the active ingredients is carried out before administration of the other or others commences.

The terms “fixed combination”, “fixed dose” and “single formulation” as used herein refers to a single carrier or vehicle or dosage form formulated to deliver an amount, which is jointly therapeutically effective for the treatment of OA, of both therapeutic agents to a patient. The single vehicle is designed to deliver an amount of each of the agents along with any pharmaceutically acceptable carriers or excipients. In some embodiments, the vehicle is a solution or a suspension. The term “non-fixed combination” or “kit of parts” means that the therapeutic agents of the combination of the invention are both administered to a patient as separate entities either simultaneously, concurrently or sequentially with no specific time limits, wherein such administration provides therapeutically effective levels of the two compounds in the body of a subject in need thereof.

### Compound 1 Demonstrates Therapeutic Efficacy

Compound 1 is a modified ANGPTL3 polypeptide that has been shown to demonstrate chondrogenic and chondroprotective effects. Compound 1 has been previously described in WO2014/138687, the contents of which are fully incorporated by reference. Without wishing to be bound by theory, it is believed that Compound 1 acts directly on cartilage resident-mesenchymal stromal cells (CR-MSCs) and articular chondrocytes through binding to α5β1 and αVβ3 integrins to transmit its anabolic repair effects on cartilage cells, promoting the formation of articular cartilage extracellular matrix proteins in mature chondrocytes and in CR-MSCs. The amino acid sequence of Compound 1 is set forth below in Table 1.

TABLE 1
Amino Acid Sequence of Compound 1

SEQ ID NO: 1
IPAECTTIYNRGEHTSGMYAIRPSNSQVFHVYCDVISGSPWTLIQHRIDGSQNFNETWENYKYGFGRL
DGEFWLGLEKIYSIVKQSNYVLRIELEDWKDNKHYIEYSFYLGNHETNYTLHLVAITGNVPNAIPENKDL
VFSTWDHKAKGHFNCPEGYSGGWWWHDECGENNLNGKYNKPRAQSKPERRRGLSWKSQNGRLY
SIKSTKMLIHPTDSESFE

In some embodiments, the Compound 1 administered according to the described dosing regimens is unmodified. In other embodiments, the Compound 1 is PEGylated. In other embodiments, the Compound 1 is fused to a heterologous peptide. In certain embodiments, the Compound 1 is fused to any one of human serum albumin (HSA), an immunoglobulin heavy chain constant region (Fc), a polyhistidine, a glutathione S transferase (GST), a thioredoxin, a protein A, a protein G, a maltose binding protein (MBP), or a fragment of any of the foregoing heterologous polypeptide(s). In particular embodiments, the heterologous polypeptide is fused at the amino-terminal end of Compound 1. In additional or alternative embodiments, the heterologous polypeptide is fused at the carboxyl-terminal end of the Compound 1. In certain other embodiments, the Compound 1 is delivered according to the drug delivery system described in US Publication Number 2020/0108153, the contents of which are fully incorporated by reference herein.

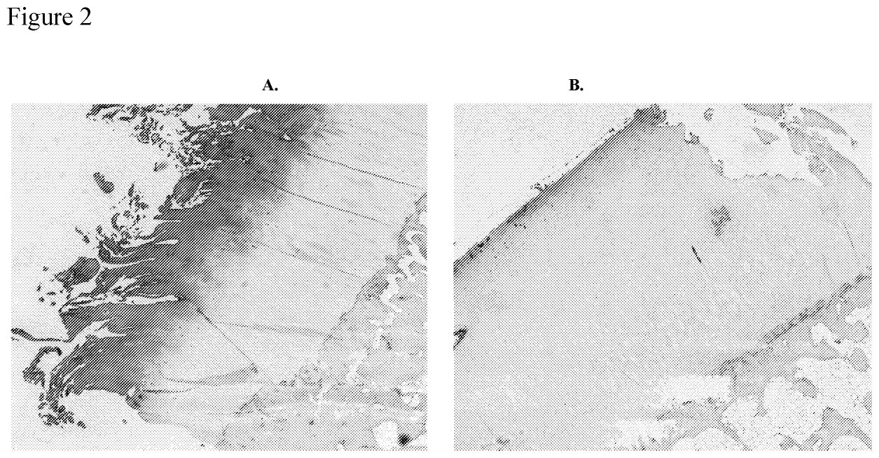

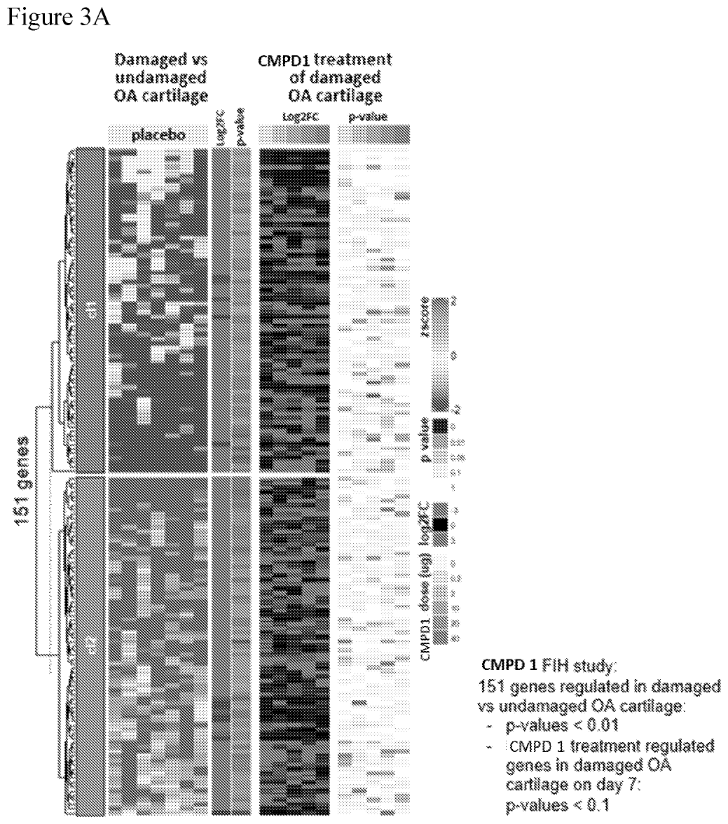

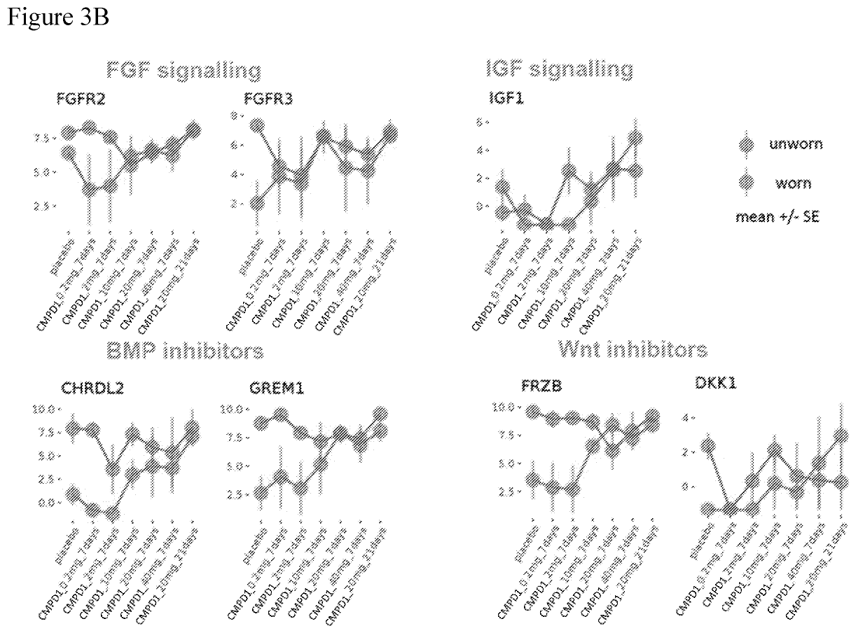

As explained above, Compound 1 has been shown to exhibit chondrogenic activity. For example, in a first-in-human (FIH) study Compound 1 was evaluated in human patients with OA who were scheduled for total knee replacement (TKR). Up to 40 mg Compound 1 was administered via intra-articular administration as a single dose 3 weeks, 1 week or 2 hours prior to surgery and safety, tolerability, pharmacokinetics (PK), and immunogenicity (IG) data were collected. Based on this study, it was determined that no significant drug-related Adverse Events (AE) or Serious Adverse Events (SAE) were reported, and Compound 1 was rapidly eliminated from the synovial fluid of the knee. Immunohistochemical analysis demonstrated that Compound 1 has a more pronounced penetration into damaged knee tissue as compared to undamaged tissue. (FIG. 2). RNASeq analysis also suggested Compound 1 modulates the activity of several genes involved in cartilage repair (FIG. 3A, B), and that this effect may last up to 21 days post-injection.

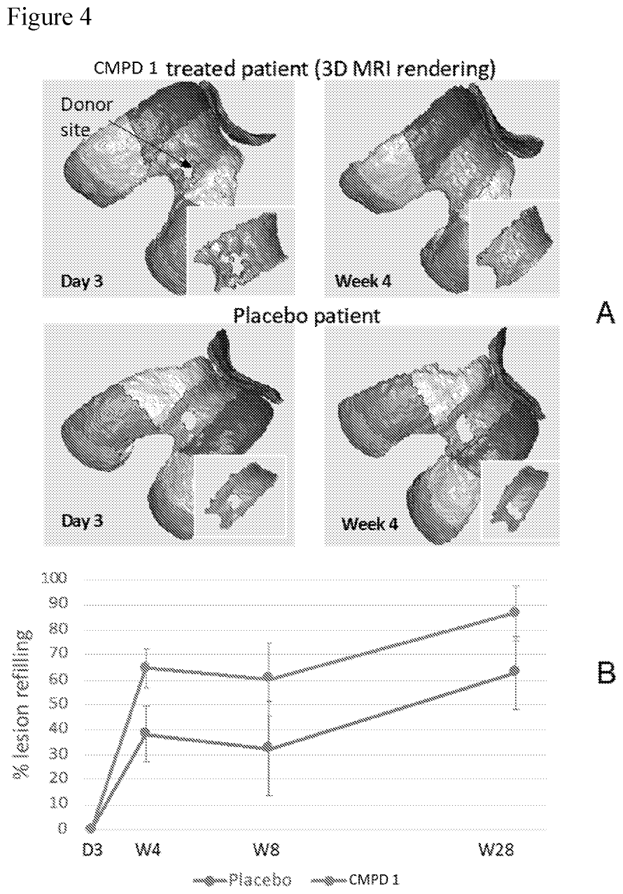

A proof of mechanism (POM) study was subsequently performed in participants undergoing autologous chondrocyte implantation (ACI) to treat a focal cartilage lesion. Those participants received a single injection of 20 mg Compound 1, and both the extent of tissue growth and the quality of tissue composition were evaluated with 7 Tesla MRI including sodium sequences. Results showed that tissue compatible with early hyaline cartilage was detected at the donor sites at 4 and 12 weeks post-Compound 1 dose. (FIG. 4, 5). No drug-related safety signal, including hypersensitivity reaction, was reported. There were no deaths or SAEs during the study and all of the AEs reported were mild to moderate in severity.

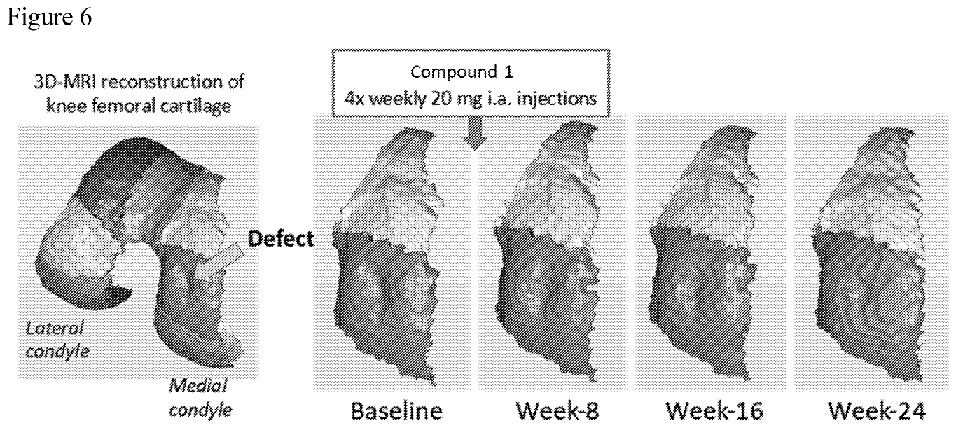

Part A of a proof of concept (PoC) study has completed dosing and follow-up in participants with knee cartilage injury who received 4 weekly injections of 20 mg Compound 1 and were then followed up for 52 weeks. Results confirm the cartilage anabolic activity of Compound 1 in humans at 28 weeks follow up measured with 3T MRI. (FIG. 6, 7). Overall, at the interim analysis, the treatment was well-tolerated and no relevant systemic safety signal was reported. Part B of the PoC study in participants with mild-moderate knee OA, receiving 4 monthly injections of 20 mg or 40 mg Compound 1, is currently ongoing.

The clinical data summarized above and described in more detail in the Examples affirm that Compound 1 demonstrates a clear dose response pattern coupled with a relatively short systemic exposure that surprisingly conveys a long pharmacodynamic effect. Compound 1 also demonstrates a favorable safety profile in human subjects. In view of this data, the inventors developed the dosing regimens disclosed herein as a means to convey a therapeutic benefit of Compound 1 while minimizing the number and frequency of injections. The disclosed dosing regimens are unexpectedly and surprisingly therapeutically effective even though the total number and frequency of doses is significantly less than previously thought necessary.

### Methods of Treatment

Provided herein are methods of treating arthritis in a subject comprising administering to a joint of the subject an intra-articular dose of Compound 1 alone or in combination with an anti-IL-1β antibody. In some embodiments, the subject has arthritis, e.g., osteoarthritis, pre-arthritic trauma-associated changes, or autoimmune arthritis. In certain embodiments, the osteoarthritis is knee osteoarthritis. In further embodiments, the subject has osteoarthritis with inflammation. In other embodiments, the individual does not have, but is at risk for, arthritis with inflammation.

In embodiments where the disease or disorder is osteoarthritis, treatment according to one of the dosing regimens described herein is expected to slow or halt the progress of OA and reduce or eliminate symptoms associated with osteoarthritis as compared to treatment with placebo. In one non-limiting example, treatment may reduce pain as measured by the Knee Injury and Osteoarthritis Outcome Score (KOOS), the Western Ontario and McMaster Universities Osteoarthritis Index (WOMAC) score, or other art recognized methods of gauging pain reduction. In another embodiment, treatment may maintain or increase a patient's quality of life or activities of daily living as measured by the osteoarthritis knee and hip quality of life questionnaire (OAKHQOL), the WHO Quality of Life-BREF, Physical Activity Scale for the Elderly, or other art recognized methods. In another embodiment, treatment may result in the maintenance or regeneration of articular cartilage, ligaments, or tendons as measured using quantitative MRI, qualitative MRI, histology of biopsies, inspection during arthroscopy, or other art recognized methods of ascertaining changes in joint tissue. In other embodiments, treatment may result in the maintenance of joint structure based on cartilage volume as determined by quantitative MRI. In another non-limiting example, treatment according to one of the presently described dosing regimens may improve or maintain (e.g., prevent further decrease) the function in the affected joint as assessed through WOMAC function, KOOS score, reduction in stiffness, or other art recognized methods of assessing physical function. In other embodiments, increase in performance-based physical function can be assessed by a 40-meter (4×10 m) fast-paced walk test, 30-second chair stand test, 6-minute walking test, gait analysis, activity measures, or other art recognized methods. In another non-limiting example, treatment according to one of the presently described dosing regimens may prolong the survival of the joint affected with OA and/or increase the subject's quality of life. In yet another non-limiting example, treatment according to a dosage regimen of the present invention may prevent or delay the need for joint replacement surgery. In other embodiments, treatment may be effective in reducing synovitis or bursitis.

The therapeutic compounds may be administered according to any known administration method. In certain preferred embodiments, the therapeutic compounds are administered via intra-articular administration. Other possible routes of administration include, e.g., intradermal, intramuscular, intravenous, and sub-cutaneous. The therapeutic compounds may also be administered according to any known means for administering a therapeutic to a patient, including, but not limited to, a pre-filled syringe, a vial and syringe, an injection pen, an autoinjector, an i.v. drip and bag, a pump, a patch pump, etc. With such items, a patient may self-administer the drug (i.e., administer the drug on their own behalf) or a physician may administer the drug.

### Dosing Regimens

In some embodiments, Compound 1 may be administered according to the dosage regimens described herein. The most effective dosages and dosage regimens for an individual subject may depend on the specific disease or condition to be treated and its severity. The dosing regimens may be continued or repeated until there is no longer a therapeutic benefit to the subject.

In some embodiments, the dosing regimen for Compound 1 may comprise one or more dosing cycles. Each dosing cycle may comprise one or more months, e.g., one month, two months, three months, four months, five months, six months, seven months, eight months, nine months, ten months, eleven months, or twelve months. A twelve month dosing cycle may also be referred to as a yearly dosing cycle. In preferred embodiments, the dosing cycle is a six-month or twelve-month (yearly) dosing cycle.

Each dosing cycle may comprise the administration of one or more, e.g., two, three, four, five, six, seven, eight, nine, ten, eleven, twelve, thirteen, fourteen, fifteen, sixteen, seventeen, eighteen, nineteen, twenty, twenty-one, twenty-two, twenty-three, or twenty-four, doses of Compound 1 during the dosing period. In embodiments comprising the administration of two or more doses during the dosing period, the doses may be administered during consecutive periods, e.g., consecutive days, weeks, or months. For example, in a dosing cycle comprising consecutive administration of four doses of Compound 1, each dose may be administered once a week for four consecutive weeks, or once a month for four consecutive months. Alternatively, the two or more doses may be administered in alternating periods of time, e.g., every other day, week, or month. For example, in a dosing cycle comprising alternate administration of four doses of Compound 1, each dose may be administered every two weeks (bi-weekly) for two months.

In some embodiments, each dosing cycle comprises the same number of doses. For example, in a dosing regimen comprising two dosing cycles, each dosing cycle may comprise three administrations of Compound 1. In other embodiments, the number of doses administered may vary from dosing cycle to dosing cycle within the same dosing regimen. For example, in a dosing regimen comprising three dosing cycles, dosing cycle 1 may comprise the administration of three doses of Compound 1 and dosing cycles 2 and 3 may comprise the administration of one dose of Compound 1.

In embodiments comprising two or more doses, the resting period does not begin until the final dose of the dosing period has been administered. For example, in a one month dosing cycle comprising two doses administered in alternate weeks, the doses could be administered in week one and week three, and the resting period could be week four. In some embodiments, the resting period is of the same period of time as the dosing period. For example, if the dosing period is one month then the resting period is one month. In other embodiments, the resting period is of a different period of time than the dosing period. For example, if the dosing period is one month then the resting period may be one week or two months. In some embodiments, the length of the dosing period and/or the length of the resting period may vary from dosing cycle to dosing cycle. For example, in a dosing regimen comprising two six-month dosing cycles, dosing cycle 1 may comprise a three-month dosing period and a three-month resting period, and dosing cycle 2 may comprise a one-month dosing period and a five-month resting period.

In a preferred embodiment, an Compound 1 dosing regimen comprises one or more six-month dosing cycles comprising one dose (i.e., a one-month dosing period) followed by a five-month resting period. In an alternative preferred embodiment, the six-month dosing cycle comprises three doses administered once a month for three consecutive months (i.e., a three-month dosing period) followed by a three-month resting period. In another preferred embodiment, the Compound 1 dosing regimen comprises one or more twelve-month dosing cycles comprising one dose (i.e., a one-month dosing period) followed by an eleven-month resting period. In an alternative preferred embodiment, a twelve-month dosing cycle comprises three doses administered once a month for three consecutive months (i.e., a three-month dosing period) followed by a nine-month resting period.

In one embodiment, an Compound 1 dosing regimen comprises at least four six-month dosing cycles, wherein each six-month dosing cycles comprises three doses of Compound 1 administered once a month for three consecutive months followed by a three-month rest period. In another embodiment, an Compound 1 dosing regimen comprises at least eight six-month dosing cycles, wherein dosing cycles 1-4 (i.e., the first four dosing cycles) comprise three doses of Compound 1 administered once a month for three consecutive months followed by a three-month rest period, and dosing cycles 5-8 comprise one dose of Compound 1 followed by a five-month rest period.

In another embodiment, an Compound 1 dosing regimen comprises four six-month dosing cycles, wherein each six-month dosing cycles comprises one dose of Compound 1 followed by a five-month rest period. In another embodiment, an Compound 1 dosing regimen comprises at least eight six-month dosing cycles, wherein each six-month dosing cycle comprises one dose of Compound 1 followed by a five-month rest period.

In another embodiment, an Compound 1 dosing regimen comprises at least two twelve-month dosing cycles, wherein three doses are administered once a month for three consecutive months followed by a nine-month resting period. In another embodiment, an Compound 1 dosing regimen comprises at least four twelve-month dosing cycles, wherein dosing cycles 1 and 2 comprise three doses are administered once a month for three consecutive months followed by a nine-month resting period, and dosing cycles 3 and 4 comprise one dose of Compound 1 followed by an eleven-month resting period.

### Dose Amounts

In some embodiments, the dose of Compound 1 administered via intra-articular injection is about 10-100 mg, about 10-90 mg, about 10-80 mg, about 10-70 mg, about 10-60 mg, about 10-50 mg, about 10-40 mg, about 10-30 mg, about 10-20 mg, about 20-100 mg, about 20-90 mg, about 20-80 mg, about 20-70 mg, about 20-60 mg, about 20-50 mg, about 20-40 mg, about 20-30 mg, about 30-100 mg, about 30-90 mg, about 30-80 mg, about 30-70 mg, about 30-60 mg, about 30-50 mg, about 30-40 mg, about 40-100 mg, about 40-90 mg, about 40-80 mg, about 40-70 mg, about 40-60 mg, about 40-50 mg, about 50-100 mg, about 50-90 mg, about 50-80 mg, about 50-70 mg, about 50-60 mg, about 60-100 mg, about 60-90 mg, about 60-80 mg, about 60-70 mg, about 70-100 mg, about 70-90 mg, about 70-80 mg, about 80-100 mg, about 80-90 mg, or about 90-100 mg.

In other embodiments, the dose of Compound 1 administered via intra-articular injection is about 10-55 mg, about 10-45 mg, about 10-35 mg, about 10-25 mg, about 10-15 mg, about 15-60 mg, about 15-55 mg, about 15-50 mg, about 15-45 mg, about 15-40 mg, about 15-35 mg, about 15-30 mg, about 15-25 mg, about 15-20 mg, about 20-55 mg, about 20-45 mg, about 20-35 mg, about 20-25 mg, about 25-60 mg, about 25-55 mg, about 25-50 mg, about 25-45 mg, about 25-40 mg, about 25-35 mg, about 25-30 mg, about 30-55 mg, about 30-45 mg, about 30-35 mg, about 35-60 mg, about 35-55 mg, about 35-50 mg, about 35-45 mg, about 35-40 mg, about 40-55 mg, about 40-45 mg, about 45-60 mg, about 45-55 mg, about 45-50 mg, about 50-55 mg, or about 55-60 mg.

In other embodiments, the dose of Compound 1 administered via intra-articular injection is about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In preferred embodiments, the dose of Compound 1 administered is 20-40 mg, 20 mg, or 40 mg.

In some embodiments, the dose of Compound 1 administered via intra-articular injection is 10-100 mg, 10-90 mg, 10-80 mg, 10-70 mg, 10-60 mg, 10-50 mg, 10-40 mg, 10-30 mg, 10-20 mg, 20-100 mg, 20-90 mg, 20-80 mg, 20-70 mg, 20-60 mg, 20-50 mg, 20-40 mg, 20-30 mg, 30-100 mg, 30-90 mg, 30-80 mg, 30-70 mg, 30-60 mg, 30-50 mg, 30-40 mg, 40-100 mg, 40-90 mg, 40-80 mg, 40-70 mg, 40-60 mg, 40-50 mg, 50-100 mg, 50-90 mg, 50-80 mg, 50-70 mg, 50-60 mg, a 60-100 mg, 60-90 mg, 60-80 mg, 60-70 mg, 70-100 mg, 70-90 mg, 70-80 mg, 80-100 mg, 80-90 mg, or 90-100 mg.

In other embodiments, the dose of Compound 1 administered via intra-articular injection is 10-55 mg, 10-45 mg, 10-35 mg, 10-25 mg, 10-15 mg, 15-60 mg, 15-55 mg, 15-50 mg, 15-45 mg, 15-40 mg, 15-35 mg, 15-30 mg, 15-25 mg, 15-20 mg, 20-55 mg, 20-45 mg, 20-35 mg, 20-25 mg, 25-60 mg, 25-55 mg, 25-50 mg, 25-45 mg, 25-40 mg, 25-35 mg, 25-30 mg, 30-55 mg, 30-45 mg, 30-35 mg, 35-60 mg, 35-55 mg, 35-50 mg, 35-45 mg, 35-40 mg, 40-55 mg, 40-45 mg, 45-60 mg, 45-55 mg, 45-50 mg, 50-55 mg, or 55-60 mg.

In other embodiments, the dose of Compound 1 administered via intra-articular injection is 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, or 100 mg. In preferred embodiments, the dose of Compound 1 administered is 20-40 mg, 20 mg, or 40 mg.

### Kits

The disclosure also encompasses kits for treating a patient with OA. In some embodiments, the kits are for treating a patient with knee OA. In other embodiments, the kits are for treating a patient with knee OA with inflammation. Such kits comprise Compound 1 (e.g., in liquid or lyophilized form) or a pharmaceutical composition comprising Compound land one or more pharmaceutically acceptable carriers. Additionally, such kits may comprise means for administering the Compound 1 (e.g., a syringe and vial, a prefilled syringe, a prefilled pen, a patch/pump) and instructions for use. The instructions may disclose providing the Compound 1 to the patient as part of a specific dosing regimen.

The phrase “means for administering” is used to indicate any available implement for systemically administering a drug to a patient, including, but not limited to, a pre-filled syringe, a vial and syringe, an injection pen, an autoinjector, an i.v. drip and bag, a pump, patch/pump, etc. With such items, a patient may self-administer the drug (i.e., administer the drug on their own behalf) or a care-giver or a physician may administer the drug.

Disclosed herein are kits for the treatment of a patient having knee osteoarthritis with or without intra-articular inflammation, comprising: (a) a pharmaceutical composition comprising a therapeutically effective amount of Compound 1 and one or more pharmaceutically acceptable carriers; (b) means for administering the Compound 1; and (c) instructions for the intra-articular administration of Compound 1.

In one specific embodiment, a use is provided, of (a) a pharmaceutical composition comprising Compound 1 and one or more pharmaceutically acceptable carriers, and (b) means for intra-articular administration of the Compound 1 to a patient having knee OA wherein the Compound 1 is to be administered intra-articularly to the patient with a dose of about 20 to 40 mg at four week (monthly) intervals according to a dosage regimen disclosed herein.

### EXAMPLES

The following examples are intended to illustrate the invention and are not to be construed as being limitations thereon. Abbreviations are used as is conventional in the art.

### Example 1: A Randomized, Placebo Controlled, Double-Blind First-In-Human Single Ascending Dose Study of Compound 1 in Primary Osteoarthritis Patients Scheduled for Total Knee Replacement

Methods: A first-in-human, randomized, single-center, double-blind, placebo-controlled, single ascending dose trial was conducted in patients aged 50-75 years with knee OA scheduled for total knee replacement (TKR). Patients were randomized 3:1 (Compound 1 to placebo) in each of 7 cohorts consisting of 4 patients each. The 5 increasing intra-articular dose levels ranging 0.2-40 mg (0.2 mg, 2 mg, 10 mg, 20 mg, and 40 mg) were administered 7 days before TKR. Two additional 20 mg dose levels were also administered 2 hours or 21 days before TKR. Key safety parameters included AEs, injection-site reactions and detection of anti-drug antibodies against Compound 1. Knee tissues were obtained during the TKR procedure to assess local exposure to Compound 1 through immunohistochemical (IHC) staining, and RNA sequence (RNA-Seq) analysis was performed on cartilage tissue originating from visually damaged or undamaged areas of the joint surface that was removed intraoperatively.

Results: In total, 30 patients were randomized to Compound 1 (n=21) or placebo (n=7). Two patients withdrew consent prior to treatment. The mean age of recruited patients was 63 years, 68% were female (n=19), and 96% were caucasian (n=27). A total of 19 (Compound 1, n=14; placebo, n=5) patients experienced at least one AE. The overall incidence of AEs was 66.7% (14/21) for Compound 1 and 71.4% (5/7) for placebo. One drug-related case of dry mouth/dysgeusia was reported in the 40 mg cohort, which resolved spontaneously and was considered mild. Ten SAEs were reported in five patients (Compound 1, n=3; placebo, n=2), although these were considered related to the surgery and not related to Compound 1. Anti-Compound 1 antibodies were not detected in any patient. After intra-articular injection, Compound 1 was dose-dependently distributed from the joint to systemic circulation with Cₘₐₓ typically reached between 2 to 6 hours after the administration, followed by rapid elimination. IHC demonstrated that Compound 1 penetrated the articular cartilage shortly after injection (2 hours), with more pronounced penetration into the damaged areas (FIG. 2). Compound 1 was not detectable in the articular cartilage or synovial-fluid 7 days post intra-articular injection (after administration of up to 20 mg).

RNA-Seq analysis demonstrated that 151 genes were significantly up- or down-regulated in damaged versus undamaged articular cartilage samples from placebo-treated OA patients (FIG. 3A). Compound 1 counter-regulated most of these OA-regulated genes in damaged cartilage 7 days post treatment, suggesting a broad effect of Compound 1 on genes involved in OA pathogenesis. RNA-Seq analysis further demonstrated modulation of several genes involved in cartilage homeostasis and repair, suggesting a broad effect by Compound 1 up to 21 days post-injection (FIG. 3B). These effects were both dose-dependent and mostly present in the damaged cartilage tissue.

Conclusions: In this study, Compound 1 displayed a favorable safety profile without any clinically significant drug-related safety signals or immunogenicity. Compound 1 further showed a tendency to preferentially penetrate into damaged cartilage; was quickly cleared locally and systemically; and counter-regulated several cartilage genes involved in OA pathogenesis at the RNA level.

### Example 2: A Randomized, Placebo-Controlled, Patient and Investigator Blinded, Single Dose, Proof of Concept Study Investigating the Safety, Tolerability and Preliminary Efficacy of Intra-Articular Compound 1 in Regenerating the Articular Cartilage of the Knee at Donor Sites in Patients Undergoing Autologous Chondrocyte Implantation

Methods: This was a randomized, placebo-controlled, double-blind, single dose, proof-of-mechanism study in subjects with cartilage lesions undergoing autologous chondrocyte implantation (ACI). In total, 14 subjects were treated with a single i.a. injection (9 in Compound 1 20 mg and 5 in placebo, 2:1 randomization ratio) that was administered at the end of the first surgical procedure. The study was designed to assess cartilage regeneration 1) at the artificially created ACI donor site in the intercondylar notch with a full thickness cartilage defect and 2) at the index lesion cartilage lesions (defect site). Spontaneous repair was minimized by avoiding breaching the bone lamina while performing the biopsy. Assessments of the treatment effects were performed at Day 3 (baseline), Week 4 (primary endpoint), Week 12 and Week 28 using 7T MRI to detect early signs of cartilage matrix production both at the donor site and the defect site, along with histological confirmation at week 4 (2nd ACI treatment arthroscopy). The index lesion to be treated with ACI was assessed with MRI only at Day 3 and Week 4, prior to the implantation of the chondrocyte graft. Volumes of the donor site and the cartilage sub-region containing the main lesion, as well as their glycosaminoglycan content (GAG), were measured by 7 Tesla high-resolution morphological (proton)-MRI and indirectly by sodium-MRI, respectively. While the volume of the donor site was measured via manual segmentation of the 3D proton images, the cartilage sub-region volume containing the main lesion, whose shape is by nature more complex than the surgically created lesion, was measured via an automated segmentation approach using a 3D-active shape model. All sodium-MRI measurements were performed using a 15-channel sodium-only knee array coil and images with a resolution of 1.5×1.5×3 mm³ were obtained in 25 min of scanning time. For region of interest (ROI) analyses, sodium concentration maps were rescaled to the resolution of morphological proton images and overlaid with the corresponding morphological image. Cartilage sodium concentrations were calculated by using a calibration curve obtained for each scan from agarose phantoms having different sodium concentrations. GAG content in the index region was normalized to that of corresponding healthy regions of the same knee. During the second surgical procedure on Week 4, a biopsy of the regenerated tissue was taken at the donor site, and tissue debris from the defect site was collected for histological and immunohistochemical analysis prior to the ACI graft implantation.

Results: The i.a. injection of Compound 1 resulted in a 65±8% refilling (vs placebo: 38±11%, p=0.04) of the donor site after 4 weeks in all treated patients (FIG. 4A) and increased to 86±11% at Week 28 (vs placebo: 63±14%, p=0.12) (FIG. 4B). In two placebo patients, a partial refilling of the donor site was seen at Week 4, but it was not maintained at Week 12 and therefore considered a blood clot after a potential lesion in the osseous lamina that had been absorbed at Week 12. Similarly, partial repair of the main cartilage lesion was observed at Week 4, prior to the ACI graft implantation (change from baseline in the volume of the sub-region encompassing the defect—Compound 1: +128±97 mm³ vs placebo: +16±30 mm³, p=0.03).

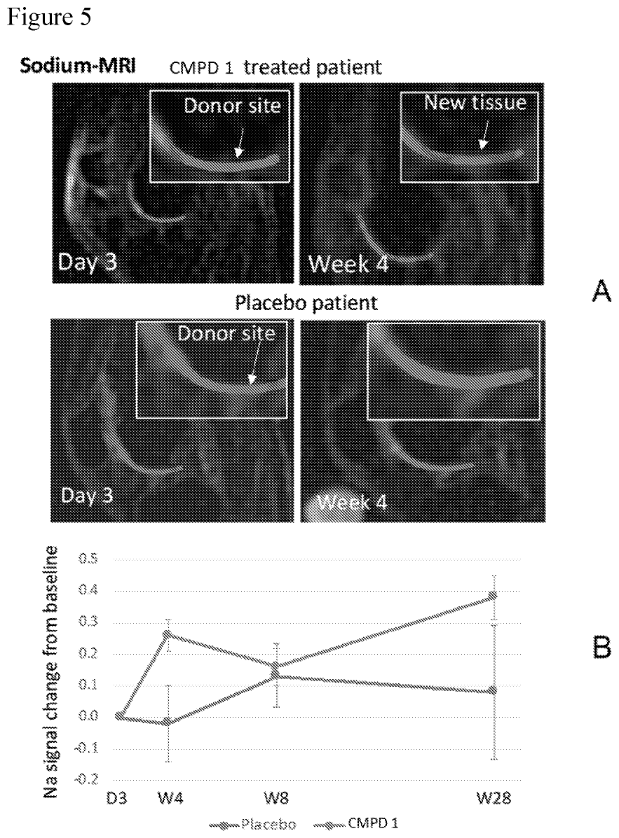

Sodium-MRI confirmed the hyaline-like cartilage nature of the regenerated tissue in the donor site: The sodium signal in the donor site increased by 26±5%, 16±6% and 38±7% in the Compound 1 group vs. −2±12% (p=0.12), 13±10% (p=0.51) and 8±21% (p=0.15) in the placebo group at Weeks 4, 12 and 28, respectively indicating increasing GAG content in the Compound 1 group (FIG. 5B). Post-hoc pooled analysis of the sodium MRI data from both the donor and defect sites showed a statistically significant (p=0.01) increase in sodium signal intensity at Week 4.

Histological and immunohistochemical assessments of biopsies taken at the donor site on Week 4 early demonstrated features of hyaline cartilage in the regenerated tissue of Compound 1-treated patients, as suggested by semi-quantitative International Cartilage Regeneration & Joint Preservation Society (ICRS) II histological scoring, and by collagen type 2 staining.

Compound 1 was rapidly distributed from the joint to the systemic circulation and no drug-related AEs or SAEs were reported during the course of this study.

Conclusions: A single i.a. injection of Compound 1 at 20 mg promoted refilling of the biopsy donor site with a full thickness cartilage defect in patients undergoing an ACI procedure. The newly regenerated cartilage tissue at the donor site appeared of hyaline-like quality as evidenced from its enriched content in proteoglycans detected by sodium MRI (FIG. 5A). Exploratory assessment of the index lesion, prior to grafting 4 weeks after the i.a. injection of Compound 1, also showed signs of tissue formation from filling of the lesion. Finally, Compound 1 displayed a consistent systemic pharmacokinetic profile together with a favorable safety profile with no significant drug related safety signals and no immunogenicity.

### Example 3: A Two-Part, Randomized, Placebo-Controlled, Patient and Investigator Blinded, Study Investigating the Safety, Tolerability and Preliminary Efficacy of Intra-Articular Compound 1 Injections in Regenerating the Articular Cartilage of the Knee in Patients with Articular Cartilage Lesions (Part A) and in Patients with Knee Osteoarthritis (Part B)

Part A—Methods: This is a randomised, double-blind, placebo (“PBO”)-controlled, proof-of-concept study in patients with a partial thickness cartilage lesion. 58 patients (43 [20 mg Compound 1]; 15 [PBO]), stratified by lesion type (condylar or patellar) were treated with 4 weekly i.a. injections. The primary endpoint was T2 relaxation time measurement as a marker of collagen fiber network, cartilage lesion-volume was a secondary endpoint, both using 3 Tesla MRI. Assessments were performed at baseline, weeks (wks) 8, 16, 28 and 52 (the last in 23/58 patients). While lesion volume was determined from manually segmented images, the cartilage volume of 21 sub-regions spanning the entire knee was measured from 3D isotropic MR images employing an automated segmentation software (MR Chondral Health [MRCH], Siemens). The treatment effect was evaluated for the index region volume encompassing the lesion (FIG. 6).

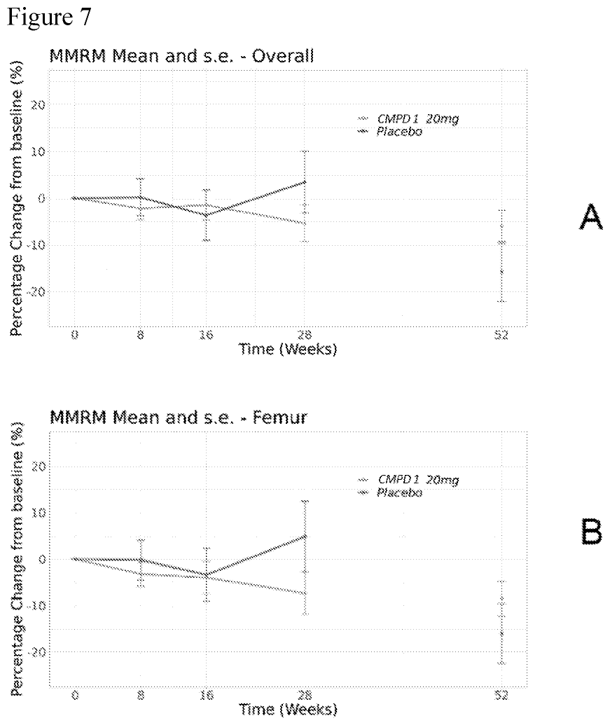

Part A—Results: There was a reduction in the cartilage defect volume at EoS (week 28), measured with high-resolution MRI (manual segmentation), in response to Compound 1. Such reduction was even more pronounced if the percentage change from baseline in cartilage defect volume was used as the response variable in the MMRM model (FIG. 7). Particularly, the one-sided p-value related to the difference between Compound 1 and placebo at week 28 was 0.08 (vs. PBO) for the sub-group of patients with a femoral lesion (the p-values at weeks 16 and 53 were 0.47 and 0.85 respectively). In contrast, no sign of defect filling could be detected in the subgroup of patients with a patellar lesion, no change in T2 relaxation time values was detected between treatment and PBO groups. Given the limitations of measuring small, irregularly-shaped lesions with manual image-analysis, the MRCH approach was used (FIG. 6) and Compound 1-induced refilling of the cartilage lesions in patients with femoral lesions was detected (Δ=96 mm³ at wk 16). Limiting the analysis to patients with condylar lesions only, the benefit of Compound 1 appeared to be maintained at wks 28 (Δ=68 mm³) and 52 (Δ=117 mm³). No overgrowth was detected in the lateral femoral condyles without cartilage damage.

The overall safety profile was positive with treatment emergent mild/moderate local reactions (incidence of joint swelling [9.3% vs 0%] and arthralgia [7.0% vs 6.7%] for Compound 1 vs PBO) resolving spontaneously or with paracetamol/NSAIDs. No anti-drug antibodies were detected.

Part A—Conclusion: Treatment with 4 weekly i.a. injections of 20 mg Compound 1 resulted in regeneration of damaged cartilage in patients with femoral articular cartilage lesions. Automated measurement of cartilage volume in the femoral index region was able to detect a relevant treatment effect and was found to be more sensitive than the manual segmentation method. No sign of cartilage overgrowth was observed in healthy femoral regions. Compound 1 showed a favorable safety and tolerability profile.

Part B—Methods: This is a randomised, double-blind, placebo (PBO)-controlled, proof-of-concept study in patients with mild to moderate osteoarthritis (Kellgren and Lawrence (K&L) grade 2-3 and joint space width 2-4 mm). 75 patients (25 [40 mg Compound 1], 25 [20 mg Compound 1]; 25 [PBO]), are treated with a total of 4 i.a. injections over the period of 4 months. The primary endpoint is safety and tolerability as well as the change in cartilage volume/thickness in the index region at Week 28 and 52 using 3 Tesla MRI. Cartilage quality will be assessed as secondary endpoint using T2 relaxation times as surrogate marker. In addition, pain and function are evaluated using the KOOS as secondary endpoints. Further exploratory endpoints comprise PK/PD assessments, biomarker, protein-expression and gene analyses. Assessments are performed at baseline, weeks 8, 16, 28 and 52. While lesion volume is determined from manually segmented images, the cartilage volume of 21 sub-regions spanning the entire knee is measured from 3D isotropic MR images employing an automated segmentation software (MR Chondral Health [MRCH], Siemens). The treatment effect is evaluated for the index region volume encompassing the lesion.

Part B—Results: Cartilage thickness and volume are expected to increase compared to baseline in the treatment groups, while a stable course or deterioration is expected in placebo patients. Pain and function are expected to improve. Data is currently insufficient to predict a dose dependent effect for Compound 1. The safety profile so far is favorable.

### Example 4: A 5-Year, Randomized, Double-Blind, Placebo-Controlled, Multi-Center Study Assessing the Efficacy, Safety, and Tolerability of Intra-Articular Regimens of Compound 1 Versus Placebo in Patients with Symptomatic Knee Osteoarthritis (ONWARDS)

The purpose of this study is to assess the efficacy, safety and tolerability of different doses (20 mg and 40 mg), number of injections in a dosing cycle (1 vs 3) and dosing cycle frequency (every 6 months vs every 12 months) of i.a. Compound 1 to determine an optimal dosing regimen for the treatment of knee OA. This study will demonstrate that dosing of Compound 1 leads to structural changes, symptomatic relief and improvement in function/quality of life of participants with knee OA.

Study Design: The study is a 2-period, multicenter, randomized, parallel-group, double-blind, placebo-controlled Phase IIb study consisting of a 2-year Core Period, followed by a 3-year Extension Period (of 2-year treatment and 1-year follow-up) aiming to assess the short and long term efficacy, safety and tolerability of multiple intra-articular regimens of Compound 1 versus placebo. Participants with radiographically determined K&L grades 2 or 3 and moderately to severely symptomatic knee OA in the target knee as indicated by a WOMAC pain score of 20-45 on a 0-50 scale prior to initiating study drug will be enrolled.

This study is aimed at defining the optimal dose regimen of i.a. Compound 1 in moderately to severely symptomatic (WOMAC Pain 20-45) knee OA (K&L 2-3) participants. To this purpose, the study will test two dose levels (20 mg and 40 mg), two dose frequencies and two cycle frequencies as follows:
Compound 1 20 mg Q4w×3, Cycle every 6 months
Compound 1 40 mg×1 and placebo Q4w×2, Cycle every 6 months
Compound 1 40 mg Q4w×3, Cycle every 12 months
Compound 1 40 mg Q4w×3, Cycle every 6 months
Placebo Q4w×3, Cycle every 6 months
   The regimens in this study will allow for comparisons of safety and efficacy data to: i) determine the appropriate dose (20 mg vs 40 mg) using the 20 mg Q4w×3 every 6 months vs 40 mg Q4w×3 every 6 months; and ii) extrapolate for the optimal dosing regimen using 40 mg×1 every 6 months, 40 mg Q4w×3 every 12 months and 40 mg Q4w×3 every 6 months.

On Week 0 (Randomization Visits) of the Core Period, eligible participants will be randomized to one of the five treatment groups at a 1:1:1:1:1 allocation and will receive a 4 mL i.a. injection of either Compound 1 20 mg or 40 mg or placebo (FIG. 1). Approximately 550 participants will be randomized.

During the Core Period, all participants will receive i.a. injections of Compound 1 or placebo (Q4w×3) every 6 months for 2 years. Compound 1 treatment arms will receive active Compound 1 20 mg Q4w×3 every 6 months, 40 mg×1 every 6 months, 40 mg Q4w×3 every 12 months, or 40 mg Q4w×3 every 6 months administered i.a. to the target knee. Participants randomized to placebo will receive i.a. injections of saline solution for injection Q4w×3 every 6 months in the Core Period. To maintain the study blind, i.a. Compound 1 or placebo injections will be administered to keep the same number of total injections (Q4w×3 every 6 months in the Core Period and once every 6 months for 2 years in the Extension Period) in all cohorts. At the end of the Core Period, participants will enter the Extension Period.

During the Extension Period, all participants will receive one i.a. injection of Compound 1 or placebo every 6 months for 2 years. Participants who received active Compound 1 every 6 or 12 months in the Core Period will receive one injection of the same dose of active Compound 1 correspondingly in the Extension Period (every 6 or 12 months, respectively). Placebo injections will be used throughout the study in order to keep the same number of injections for all arms and ensure blinding, based on the assigned frequency of treatment. Participants in at least one of these four Compound 1 arms may be discontinued from the Extension Period based on efficacy and safety results from the Week 104 primary endpoint database lock. Participants who received placebo during the Core Period will continue to receive placebo every 6 months for the Extension Period. The last 1 year of the Extension Period will be a no-treatment follow-up for all arms.

Standard of care treatments and medications for knee OA pain are allowed throughout the study.

In each cycle, intervals of consecutive study treatment injections should be 4+/−1 weeks. After Week 26, the date of first study treatment in each cycle should be calculated based on the Week 0 (randomization visit) date, and it can be +4 weeks from the calculated date; e.g. Week 26 date should be determined as 26 weeks after the Week 0 visit date, and study treatment at Week 26 should be between 26 and 30 weeks after the Week 0 date.

The study has been designed as participant- and investigator-blinded in order to reduce bias from both investigators and participants, so that a difference between the treated and control groups can be interpreted as an effect of study treatment.

Study Population: The study population will be comprised of male and female participants 40 to 75 years old with predominantly unilateral medial radiographic K-L grade 2-3 knee osteoarthritis and moderate to severe pain in the index knee (WOMAC Pain of 20-45 out of 50 on no medication or after pain medication washout) in the target knee during the screening period. A total of approximately 550 participants will be randomized. Participant selection is to be established by applying all eligibility criteria at Screening Visits 1 and 2. A relevant record (e.g., checklist) of the eligibility criteria must be stored with the source documentation at the study site. Participants who are randomized and fail to start treatment, e.g., participants randomized in error, will be considered as mis-randomized. Participants who have been randomized once cannot be screened or randomized again.

Results: Based on available clinical data from the completed and ongoing studies, a cartilage anabolic effect resulting in regeneration of the articular cartilage is expected in knee OA participants. Based on the positive results from early phase studies, a disease modifying effect is anticipated, although to be confirmed in the knee OA K&L 2-3 population. The overall risk to knee OA participants in this study is expected to be low due to the safety profile observed in completed clinical trials.

The primary objective will be to assess the efficacy of each regimen of Compound 1 compared to placebo at Week 104 as measured by the mean change from baseline in cartilage thickness of the central medial tibiofemoral compartment (cMTFC) using quantitative magnetic resonance imaging (qMRI) of the target knee. It is anticipated that treatment with Compound 1 will result in an increase in cartilage thickness as compared to placebo.

The study will also assess the efficacy of each regimen of Compound 1 compared to placebo at week 104 in meeting several secondary objectives:
Changes in baseline OA pain in the target knee will be evaluated. The endpoint for this objective will be assessed using WOMAC pain and WOMAC pain walking on flat surface item. It is anticipated that treatment with Compound 1 will result in a decrease in OA pain in the target knee as compared to placebo.
Changes in baseline in physical function will be evaluated. The endpoint for this objective will be assessed using WOMAC function. It is anticipated that treatment with Compound 1 will result in an increase in physical function as compared to placebo.
Structural changes in the total, medial and lateral tibiofemoral compartments (TFCs) in the target knee will be evaluated. The endpoint for this objective will be assessed by evaluating change in cartilage thickness using qMRI. It is anticipated that treatment with Compound 1 will result in an increase in cartilage thickness in the total, medial and lateral TFCs in the target knee as compared to placebo.
Changes in performance-based physical function will be evaluated. The endpoint for this objective will be assessed using a 40-meter (4×10 m) fast-paced walk test, 30-second chair stand test, and 6-minute walking test. It is anticipated that treatment with Compound 1 will result in an increase in performance-based physical function compared to placebo.
Changes in the proportion of patients with structural progression in the target knee will be evaluated using imaging techniques. The endpoint will be assessed by examining (1) change above the smallest detectable change (SDC) of cartilage thickness using qMRI, and (2) a loss of medial minimum joint space width (minJSW) ≥0.70 mm using x-ray. It is anticipated that treatment with Compound 1 will result in an increase in the proportion of patients with structural progression in the target knee as compared to placebo.
Safety and tolerability of the various Compound 1 regimens will be evaluated. The endpoint will be assessed using AEs and SAEs, incidence of Acute Inflammatory Reactions (AIRs) on the target knee, clinically significant changes in laboratory measures and vital signs as determined by the investigator, and the incidence of binding and neutralizing anti-drug antibodies in serum. It is anticipated that treatment will Compound 1 will prove safe and well-tolerated.

Having thus described several aspects of several embodiments, it is to be appreciated various alterations, modifications, and improvements will readily occur to those skilled in the art. Such alterations, modifications, and improvements are intended to be part of this disclosure, and are intended to be within the spirit and scope of the disclosure. Accordingly, the foregoing description and drawings are by way of example only.

Those skilled in the art will recognize, or be able to ascertain, using no more than routine experimentation, numerous equivalents to the specific embodiments described specifically herein. Such equivalents are intended to be encompassed in the scope of the following claims.

1. A method of treating knee osteoarthritis comprising administering to a human subject in need thereof one or more doses of a therapeutically effective amount of Compound 1 by intra-articular injection to a knee joint of the subject according to a dosing regimen comprising one or more dosing cycles.

2. The method of claim 1, wherein said dosing cycle is a six-month dosing cycle comprising one intra-articular injection.

3. The method of any of claims 1-2, wherein said dosing cycle is a six-month dosing cycle comprising three intra-articular injections per dosing cycle with one injection administered once a month for three consecutive months.

4. The method of claims 1-3, wherein the dosing regimen comprises at least two six-month dosing cycles.

5. The method of claims 1-4, wherein the dosing regimen comprises at least four six-month dosing cycles.

6. The method of claim 5, wherein dosing cycles 1-4 each comprise one intra-articular injection.

7. The method of claim 5, wherein dosing cycles 1-4 each comprise three intra-articular injections per dosing cycle with one injection administered once a month for three consecutive months.

8. The method of claims 1-7, wherein the dosing regimen comprises at least eight six-month dosing cycles.

9. The method of claim 8, wherein dosing cycles 1-8 each comprise one intra-articular injection.

10. The method of claim 8, wherein dosing cycles 1-4 each comprise three intra-articular injections per dosing cycle with one injection administered once a month for three consecutive months.

11. The method of claim 10, wherein dosing cycles 5-8 each comprise one intra-articular injection.

12. The method of claim 1, wherein said dosing cycle is a twelve-month dosing cycle.

13. The method of claim 12, wherein said twelve-month dosing cycle comprises three intra-articular injections per dosing cycle with one injection administered once a month for three consecutive months.

14. The method of claim 12, wherein said twelve-month dosing cycle comprises one intra-articular injection.

15. The method of claims 12-14, wherein said dosing regimen comprises at least two twelve-month dosing cycles.

16. The method of claims 12-15, wherein said dosing regimen comprises at least four twelve-month dosing cycles.

17. The method of claim 16, wherein dosing cycles 1-4 each comprise three intra-articular injections.

18. The method of claim 16, wherein dosing cycles 1-2 each comprise three intra-articular injections per dosing cycle with one injection administered once a month for three consecutive months.

19. The method of claim 18, wherein dosing cycles 3-4 each comprise one intra-articular injection.

20. The method of claims 1-19, wherein the amount of Compound 1 administered per dose is 20 mg.

21. The method of claims 1-19, wherein the amount of Compound 1 administered per dose is 40 mg.

22. The method of claims 1-21, wherein administration according to said dosing regimen results in an increase in cartilage thickness as compared to placebo.

23. The method of claims 1-21, wherein administration according to said dosing regimen results in an increase in cartilage thickness in the target knee joint.

24. The method of claim 23, wherein said increase in cartilage thickness is determined by quantitative magnetic resonance imaging (qMRI).

25. The method of claims 1-21, wherein administration according to said dosing regimen results in a decrease in osteoarthritis pain in the target knee joint.

26. The method of claim 25, wherein said decrease in osteoarthritis pain is determined by WOMAC pain and WOMAC pain walking on flat surface item.

27. The method of claims 1-21, wherein administration according to said dosing regimen results in an increase in physical function in the target knee joint.

28. The method of claim 27, wherein said increase in physical function is determined by WOMAC function.

29. The method of claims 1-21, wherein administration according to said dosing regimen results in an increase in performance-based physical function in the target knee joint.

30. The method of claim 29, wherein said increase in performance-based physical function is determined by a 40-meter (4×10 m) fast-paced walk test, 30-second chair stand test, and 6-minute walking test.

31. The method of claims 1-21, wherein administration according to said dosing regimen results in an increase in structural progression of the target knee joint.

32. The method of claim 31, wherein said increase in structural progression will be determined by qMRI and x-ray.


SEQUENCE LISTING (1 sequence)

SEQ ID NO: 1 - protein, 219 aa, Artificial Sequence
  Description of Artificial Sequence Synthetic polypeptide
IPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAQSKPERRRGLSWKSQNGRLYSIKSTKMLIHPTDSESFE